(19) United States
(12) Patent Application Publication
(10) Pub. No.: US 2021/0352890 A1
(43) Pub. Date: Nov. 18, 2021

(54) BAIT COMPOSITION FOR A PEST

(71) Applicant: Falcon Life Sciences Ltd, Malvern (GB)

(72) Inventor: Simon Upson, Malvern (GB)

(21) Appl. No.: 17/316,984

(22) Filed: May 11, 2021

(30) Foreign Application Priority Data
May 12, 2020 (GB) 2006978.7

(51) Int. Cl. A01N 25/00; A01N 25/26; A01N 65/40; A01N 65/26; A01N 65/20; A01N 65/22; A01N 31/06; A01N 43/22

(52) U.S. Cl. CPC A01N 25/006; A01N 25/26; A01N 65/40; A01N 43/22; A01N 65/20; A01N 65/22; A01N 31/06; A01N 65/26

(57) ABSTRACT

A bait composition is provided for use in a bait for ingestion by a pest. The bait composition includes a poison for killing the pest, and at least one of an insect growth regulator, a larvicide and an insecticide, for minimizing the propagation of insects from the pest's carcass. The effect of pest control involving the bait on a secondary pest population may be assessed using a suitable method.

### CROSS-REFERENCE TO RELATED APPLICATIONS

This application claims the benefit of United Kingdom Patent Application No. GB2006978.7 filed on May 12, 2020. The entire disclosure of the above application is incorporated herein by reference.

### FIELD

The present invention relates to a bait and bait composition for ingestion by a pest, particularly but not exclusively a rodent pest, for minimizing the propagation of insects such as flies, particularly but not exclusively blow flies, from the pest carcass. The present invention also relates to a method of pest control using the bait.

### INTRODUCTION

As civilization has developed and urbanization has spread, various animal species have adapted to thrive alongside humans. Some of these species are considered to be pests or vermin because they cause damage and/or spread disease, and generally breed in large numbers. Some examples of mammals or birds which can be considered as pests (collectively referred to as animal pests) include rodents, possums, raccoons, foxes, pigeons, gulls, weasels, and rabbits. Some examples of insect pests or invertebrate pests include flies (such as blow flies), beetles (such as carrion beetles), mites, moths, ants, termites, and cockroaches. The definition of a pest will vary by region and by country, so these lists should not be construed as being exhaustive.

Rodents are a particularly common pest in towns and cities as well as more rural locations. The main commensal rodents that are classed as pests are Rattus norvegicus, Rattus rattus and Mus musculus, the brown rat, black rat and house mouse respectively. Rodents can spread diseases, such as Leptospirosis, Lymphocytic choriomeningitis (LCMV) and the Black Plague. Rodents can also cause structural damage to property.

Toxic bait is one of the methods used to keep pest populations under control, including rodent populations. Various forms of bait are available, including free flow grain, wax coated blocks, ‘pasta’ bait, foam contact baits and liquid bait formulations, as well as other poison concentrates and pills. All of these forms of bait contain a poison or toxin which is intended to kill the target species.

After the bait has likely been deployed for long enough to have likely killed the target population, it is prudent to search for and collect the carcass or carcasses before they begin to rot or decompose. Ideally all of the pest carcasses in human settlements would be collected and destroyed, whether death results from toxic bait or otherwise, but this is not always possible because the pests may die in an inaccessible or unknown location. This may be underground, in a loft space, a cavity, a sewer, and so on, since toxic bait does not tend to cause immediate death and the pest which ingests the toxic bait may move from the bait location in the intervening time period.

If not removed in time, unpleasant or harmful gases can be given off from a carcass during decomposition. In addition, there can be issues with secondary poisoning if other animals such as scavengers consume parts of the carcass which contain the toxic bait.

Worse still, in cases where it is not possible to remove the carcass(es), the carcass(es) can provide an ideal place for insects, such as blow flies, to lay their eggs. Whilst blow flies and other insects serve an important role in the natural world, it is problematic when they mature in a carcass in a confined space or dwelling. For example, a decomposing rat carcass can easily lead to a large swarm of hundreds or even thousands of blow flies or a large quantity of other insects, which is a major nuisance when it occurs in a building. Unfortunately, flies are expected as a natural consequence of rodent pest control. A rodent pest controller will often visit a property at least twice—first to deal with a rodent infestation, and then to deal with a fly infestation which typically involves using toxic sprays such as permethrin, aerosols, bendiocarb or fumigants.

In addition to harming the well-being of people in the vicinity, blow flies can present a serious health risk. For example, blow flies can contaminate food with species such as Escherichia coli, Streptococcus and Campylobacter. In more extreme cases, they can cause myiasis in a person or pet, where a larva consumes living tissue from its host. Secondary poisoning is also a concern for insectivorous animals which consume blow flies and/or other insects or invertebrates hatching from a poisoned rat carcass.

Note that the term ‘blow flies’ is a collective term for many species which can help to break down or decompose carcasses. For example, in the UK, Calliphora vicina and Calliphora vomitoria are two of the main species of blow flies. In other countries, species such as Cynomyopsis cadaverina, Cochliomyia macellaria, Phormia regina, Lucilia cuprina, L. illistris, L. sericata, Phaenicia sericata and C. megacephala are examples of species that are classed as blow flies.

It is an object of the present invention to reduce or substantially obviate the aforementioned problems.

### SUMMARY

According to a first aspect of the present invention, there is provided a toxic bait composition for use in a bait to be ingested by a pest, the bait composition comprising a poison for killing the pest, and one, two or all: of an insect growth regulator (IGR), a larvicide, and an insecticide, for minimizing or controlling the propagation of an insect pest from the pest's carcass. In particular, there is a provided a bait composition as set out in claim 1. Optional features are set out in the dependent claims.

This allows for pest control of a target pest animal, such as a rodent, and at the same time allows for pre-emptive pest control of an insect pest. Where the IGR and/or larvicide are present, this substantially prevents adult insects from forming, which may in turn prevent contamination of a surface or a nuisance in a building or its surrounding area. Where the insecticide is provided, this can kill the adult insects without further pest control intervention. The different life stages of the insect pest can be targeted as deemed most appropriate for a particular infestation.

When used in rodent pest control, for example, the invention can substantially prevent blow fly or other insect infestations resulting from rodent carcasses which contain constituents of the toxic bait. This can significantly reduce the amount of harmful fly spray or other knock down chemicals used in pest control, and may avoid collateral deaths in non-target species due to the specificity of the insect growth regulator or larvicide. In addition, by minimizing or stopping insect maturation, or killing the insect soon after maturation, it also mitigates collateral damage to other creatures by substantially preventing accumulation of the initial poison(s) further up the food chain, such as in insectivorous birds for example.

Considering rodent pest control in particular, the global market for rodenticide is predicted to be in excess of USD$900 million by 2025. Tackling secondary pests such as blow flies in the same step as the primary pest by using the present invention, i.e. in a single step baiting regime, would revolutionize the industry.

The term “insect growth regulator” (abbreviated as IGR) may include or otherwise be referred to as juvenile hormone, juvenoid, ecdysteroid, growth hormone mimic and third generation insecticide.

In general, an IGR is a chemical or compound which affects insect development, typically prior to pupation. For example, an IGR may act as a larval stage interrupter which stops larvae developing to adulthood through metamorphosis. In some cases, an IGR may mimic or interrupt the effects of naturally occurring growth hormones in insects. In other cases, an IGR may act as a chitin synthesis inhibitor for larvae, for example IGRs such as the chemicals diflubenzuron and lufenuron.

Combining an animal poison and an IGR and/or larvicide means that the primary target pest is killed and the secondary target pest (an insect pest) such as a fly species will generally not hatch from any larvae or pupae in the pest carcass. The IGR can in particular prevent blow fly larvae from maturing into flies. This is because metamorphosis is a complex process, and disrupting natural hormone levels even slightly can cause it to go awry. IGRs often act on the imaginal discs, which are parts of larvae that would normally develop into adult body parts such as the head, limbs and sexual organs. By affecting the third or fourth instar (or last larval stages), or inhibiting chitin synthesis, this can cause delayed metamorphosis or pupation, incomplete metamorphosis and fatality for an insect whether in the larval stage, pupal stage or shortly after hatching as an adult.

Any adult insect that does still manage to hatch is typically disadvantaged in terms of its ability to breed, ability to move or fly, and its longevity in general. This limits the spread of the bait constituents to other non-target species, minimizing potential collateral environmental, health and hygiene issues, as well as potential reputational damage.

Therefore, any larvae or maggots present in a pest carcass containing the bait composition may fully or predominantly decompose the rodent carcass prior to pupation, but the IGR prevents them from developing into adult insects. Allowing the larvae or maggots to decompose the carcass is preferable to breakdown by bacteria and fungi, which would cause more unpleasant odors.

Where a larvicide is used, this targets an earlier stage of the insect development process, rather than disrupting the maturation of insect into an adult in the way that an IGR does. The larvicide may be a slow-acting larvicide, or provided at a low enough dose, so that larvae in the pest carcass have time to at least partly contribute to its decomposition.

Where an insecticide is used, adult insects which mature in the carcass may die prior to, during or soon after hatching, due to exposure to the insecticide.

The bait composition is ideal for use in a building such as a tower block, a hospital, a house or a farm building, such as a grain warehouse or store. The bait composition may be used in a kitchen, attic or basement, for example, which may disproportionately attract pests for food or shelter. It may also be used in structures such as an underground station, a sewer or a drain, or any other structure (or cavity therein) which may have a pest infestation.

It should be noted that the IGR may be an insect deterrent. Thus, including an IGR in the bait composition can improve the longevity of the bait for the initial or target pest, by reducing insect consumption of the bait.

The poison may be a slow-acting poison, for example killing the pest after a number of hours or days. This mitigates the development of bait avoidance, which can sometimes be an issue when using secondary rodenticides in the control of rodent populations, for example.

The IGR and/or larvicide may be present in an amount which is non-toxic to the pest ingesting the composition. The IGR chemical and/or larvicide chemical may also have a pleasant odor. Thus, the presence of the IGR and/or larvicide does not negatively affect the palatability of the bait composition for the pest intended to ingest the bait, and so the bait is effective against both of the target pests.

The pest may be a mammalian pest or a bird pest. In particular, the pest may include a rodent or rodent species. The insects or insect pest may include flies (particularly blow flies). It will be appreciated, however, that any other mammalian/bird pest and/or insect pest—such as those named in the background section above—may be selected according to the need for pest control in a particular area or location. The choice of the particular compound or combination of compounds may be influenced or determined by the insect pest known to be most commonly involved in the decomposition of a particular mammal or bird pest, once dead, in a particular region or country.

The poison may include a rodenticide. The term “rodenticide” is intended to mean any compound, composition or formulation which is toxic or poisonous to a rodent, particularly when ingested (either directly or by grooming).

The poison may be selected for one or more various target pests or creatures such as rats, mice, possums, other mammals, birds and other vertebrates. The poison may be independently selected to include one of, or a combination of, the following: alphachloralose; aluminium phosphide; brodifacoum; bromadiolone; bromethalin; calcium phosphide; chlorophacinone; cholecalciferol; coumachlor; coumafen; coumatetralyl; cyanide; difenacoum; difethialone; diphacinone; flocoumafen; fluoroacetamide; phosphorus; sodium fluoroacetate; strychnine; thallium; warfarin; zinc phosphide.

It will be appreciated that the above poisons are examples of poisonous substances for different pests, and any other suitable poison or poisons may be provided. For example, one or more various phosphides or cyanides may be used.

The IGR is preferably stable for ingestion and subsequent absorption into the pest's body, which may include absorption into the organs. The IGR may be considered to be a feed-through IGR. A feed-through is a treatment that is administered orally, either in food, in bolus form or in a water source for the target animal. The feed through may be released into the pest's feces, but some will still remain in the gastric tract and/or organs of the pest animal. Even where the IGR remains predominantly in the gastric tract, this can be sufficient to substantially minimize or control the number of flies which result from a pest carcass.

The IGR or combination of IGRs may be selected according to the target insect pest or species for a particular area or location. This can provide a tailored approach to pest control, taking account the susceptibility of the target pest or species to a particular IGR or combination.

The IGR may be independently selected from one or more of the following: one or more pyridines; one or more metallopeptidases or Angiotensin converting enzymes; one or more synthetic ecdysteroids; one or more ecdysteroid agonists; one or more juvenile [insect] hormone analogs; one or more insecticides with chitin synthesis inhibitory activity.

The or each IGR may be independently selected from the following group: methoprene, hydroprene, diflubenzuron, tebufenozide, RH 5849, pyriproxyfen, chromafenozide, methoxyfenozide, diofenolan, fufenozide, and lufenuron. Where methoprene is included, at least some of the methoprene may be S-methoprene. It will be appreciated that the selection of a particular IGR (whether or not listed above or later on) may depend on or be determined according to the target insect species, and that multiple different IGRs may each be effective for pest control of a particular insect species.

The or each IGR may be independently selected from the following group: Allosamidin, Altosid™ (isopropyl 11-methoxy-3, 7, 11-trimethyldodeca-2, 4-dienoate), Altozar™ (ethyl 3,7, 11-trimethyl-2,4-dodecadienoate), Azadirachtin (AzaGuard), Buprofenozin, Buprofezin (alkyl 3,7,11-trimethyl-2,4-dodecadienoates), Chlorfluazuron, Chromofenozide, Cyromazine 75W ([N-cyclopropyl-1, 3, 5-triazine-2, 4, 6-triamine]), Dichloro-dibenzoylhydrazine, Diflubenzuron (Vigilante), Fenoxycarb (Ro 13-5223), Flufenoxuron, Halofenozide, Hexaflumuron, Hydroprene (ethyl 3, 7, 11-trimethyidodeca-2, 4dienoate), Lufenuron, methoprene (isopropyl 11-methoxy-3, 7, 11-trimethyldodeca-2, 4-dienoate), Methoxyfenozide, Methyl 10, 11-epoxy-3,7-11-trimethyl-2,6-dodecadienoate), Nylar 2-(1-Methyl-2-(4 phenoxyphenoxy) ethoxyl pyridine, Pyriproxyfen (Nyguard, Nylar, Sumilarv) such as 11.23% Pyriproxyfen, S-Kinoprene, Tebufenozide, Teflubenzuron, Triflumuron, 2-[pm-(Flurophenoxy) phenoxy] ethyl ethylcarbamate (Ro16-1295), 20-hydroxyecdsyone, AC-291898, AI3-63604 (a juvenile hormone mimic), AI3-63967, AY SIR 8514 ([1-(4-trifluoro-methoxyphenyl)-3-(2-chlorobenzoyl)urea), MV-678 ([2-methoxy, 9-(p-isopropylphenyl)-2,6-dimethylnonane]), R-204-58, RH-2485, RH-5849, Ro 16-1295 (2-[p-(m-Fluorophenoxy) phenoxy] ethyl ethylcarbamate), Ro 13-5223 IE (ethyl [2-(p-phenoxyphenoxy) ethyl] carbamate), S-21149, S-31183, TH-6040, (N-(4-chlorophenyl)-N′-(2, 6-difluorobenzoyl)-urea), XRD-473.

It will be appreciated that the above IGRs are examples of insect growth regulators and any other suitable IGR or IGRs may be provided.

When ingested by the pest animal, the IGR can be distributed throughout the body, including in the organs and gastric tract. This means that any larvae in the pest's carcass and any larvae in the pest's feces are both exposed to the IGR.

The IGR may include one or more naturally-occurring or naturally-sourced components or constituents independently selected from the following group: Egyptian essential oil(s), celery, fenugreek, mustard, rosemary, anise, lettuce, and chamomile, or an extract thereof.

These potential constituents can have similar effects to synthetic IGRs, when provided in a suitable quantity to result in the required concentration of IGR in a pest carcass.

The IGR may include neem extract or azadirachtin (found in neem seeds). Azadirachtin can act as a growth regulator and as an anti-feedant, which may help prolong bait longevity by deterring invertebrates from eating the bait, for example. It may also prolong the larval period of some insects (such as blow fly larvae), although the amount required depends on the target pest and which stage of insect development is affected.

The azadirachtin or neem extract may be encapsulated for ingestion. This may help avoid bait avoidance by the target species, as neem is particularly strong smelling

The IGR may include an extract of Persian lilac or chinaberry tree (both otherwise known as Melia azedarach). Constituents of the extract may be encapsulated for ingestion.

The IGR may include an azasteroid for inhibiting sterol uptake, transport and/or use in larvae. For example, cholesterol uptake or use may be prevented or disrupted.

Insects cannot synthesize sterol and require it to produce growth hormones. By blocking the absorption or use of sterol, the development process is adversely affected. Some azasteroids can be effective when present at 1 ppm in a pest carcass. The azasteroid may kill larvae or otherwise promote failures in metamorphosis, development, pupation or adult insect emergence from the pupa.

The larvicide may be considered to be a feed-through larvicide. As for a feed-through IGR, some of the larvicide may be absorbed into the body of the pest and/or remain in the gastric tract, and some of the larvicide may be excreted in its feces.

The larvicide may include a DOPA decarboxylase inhibitor. The larvicide may include one or more of: methyl DOPA, benserazide, carbi-DOPA and methyl tyrosine.

The larvicide may be provided in a protected or encapsulated form. Once ingested by a pest animal, the larvicide may be absorbed via the pest's digestive system, although it will be appreciated that some of the larvicide may be excreted.

The larvicide may include a pathogenic fungus. For example, the pathogenic fungus may include Beauveria bassiana, although it will be appreciated that any suitable pathogenic fungus may be used. The fungus may be a feed-through fungus, intended for excretion. For example, the fungus may be a fungus like that used for as a feed-through treatment for chickens. Nonetheless, if a sufficient quantity of the fungus remained in the pest carcass, then this would act on the larval load of the carcass and mitigate the number of flies produced. The larvae may still partially or fully decompose the carcass prior to being overcome by the fungus.

If a delayed or prolonged IGR and/or larvicide release is required, or if the IGR and/or larvicide is strong-smelling and may deter consumption for example, then the IGR and/or the larvicide may be encapsulated (together or separately). For example, the IGR and/or larvicide may be held in a polymer or resinous material. The encapsulating material may be biodegradable, at least partially in the digestive system of a pest such as a rodent. This may be useful for slow-acting poisons, where death occurs a number of days after exposure.

For the avoidance of doubt, any one or more of the bait constituents may be encapsulated, whether independently or together. For example, the insecticide may be encapsulated. Encapsulation of any particular constituent is not essential, but may result in more effective pest control.

Where the bait composition includes an insecticide, this may include a compound such as a pyrethrin, permethrin, or one of the aforementioned plant extracts, for example. The insecticide should be stable enough for ingestion and digestion like the IGR and/or larvicide. By providing an insecticide, insects which do manage to mature and hatch from the pest carcass may die soon afterwards. Visiting insects may also be killed by the insecticide. The insecticide may be encapsulated in a similar manner to that of the IGR and/or larvicide.

Note that the insecticide may, if provided, be present in an amount or concentration which is not toxic to the animal which ingests the bait, but which can adversely affect the target insect pest after the animal dies. Possible accumulation of the insecticide along the food chain should be taken into account when selecting an insecticide, in order to minimize accumulation. In some cases, the poison may be an insecticide, where permissible for use in killing the target animal pest. The LD₅₀ value of the insecticide may be useful in determining the amount or concentration used. The poison may include an anticoagulant, such as difenacoum or bromadiolone or similar. This is particularly applicable where the poison includes a rodenticide. The poison or rodenticide may be provided at 0.005% w/w, for example.

The poison may also or instead include a naturally-sourced or naturally-occurring product such as calcipherol (also known as ergocalciferol) and/or cholecalciferol. Again, this is particularly applicable where the poison includes a rodenticide. Where the IGR is also selected to be a natural product like Egyptian essential oils, for example, the bait composition may be considered to be a natural toxic bait.

According to a second aspect of the invention, there is provided bait for ingestion by a pest, in which the bait comprises a bait composition according to the first aspect of the invention.

The identity of the IGR and/or larvicide and/or insecticide may be selected according to some or all of the following: the animal pest the bait is intended for, the insect pest expected to use the animal pest carcass for its larvae, and the mass of the bait to be deployed. Similarly, the amount of the IGR and/or larvicide in the bait may be selected or tailored according to one or more of the same factors. Either selection may be made according to a particular region or country where the bait is expected to be used.

The bait or composition may include a pest attractant, which may include a food such as sugar or grain.

The IGR and/or larvicide and/or insecticide may be added to, suspended or dissolved in, or otherwise infused into the bait during manufacture. This may be done at the same time as the poison is included, or in a separate preceding or subsequent step during bait formation. The IGR and/or larvicide and/or insecticide should ideally be distributed relatively evenly through the bait.

The bait may be in the form of a substantially solid body such as a pellet, a grain (such as free flow grain), or a block (such as a wax-coated block or mineral block). Any or all of the poison, the insect growth regulator, the insecticide and/or the larvicide may be distributed substantially homogenously throughout the solid body.

The bait may include a binder or carrier for the poison, IGR, insecticide and/or larvicide. The binder or carrier may be selected to be the same as the pest attractant in some cases. The binder or carrier may include a petroleum-based substance or a polymer or a salt, for example.

The bait may be provided as a liquid or as a foam. The carrier fluid or solvent for the composition may include water, for example. A pest may then readily consume the liquid to quench thirst. Using a liquid is useful where there are too many food sources, such as grain barns, where animal pests may avoid a food bait altogether. Where foam is used, this can easily collect on animal fur, for example whilst the pest is moving through its territory, and may be consumed during grooming or allogrooming (the grooming of other individuals). That is, the bait can be adapted according to social behavior of an animal pest population. This can be particularly effective against rodents, using their own social actions to poison most or all of a particular population.

The IGR and/or larvicide and/or insecticide may be provided in any suitable quantity in order to achieve the intended aim of preventing mass insect propagation, such as fly hatching. For example, the IGR may be provided in an amount which can result in a pest carcass, such as a dead rat, containing IGR at a concentration of at least around 1 ppm or 2 ppm. The specificity of a particular IGR or larvicide for the target pest means that only tiny quantities need to be present in order to have the desired effect, whilst other species are not adversely impacted.

Although the following discussion may focus on the amount of IGR, similar or corresponding amounts of larvicide and/or insecticide may be contemplated. It will be appreciated that the IGR, larvicide and/or insecticide may each be provided in a sufficient quantity to achieve the overall aim of preventing or controlling mass insect propagation.

The IGR may be present in amount of around 1 mg or more per kilogram of body tissue of the animal pest. For example, around 5 mg/kg may be suitable. The IGR may be present in an amount of up to about 10 ppm. This is well below the LD₅₀ value for the pest which will ingest the bait. For example, for a mouse, the LD₅₀ for methoprene has been shown to be around 5000 ppm for an acute dose. An IGR can thus be considered to have a negligible effect on the pest which initially ingests the bait and dies from the poison.

For solid bait, the mass of IGR or larvicide or insecticide used may depend on some or all of the efficacy of the IGR or larvicide or insecticide, the mass of the pest animal expected to consume the bait, and the extent of bait absorption into the body of a particular pest after bait ingestion. If there is an expectation or likelihood of partial bait consumption, a higher amount of IGR and/or larvicide and/or insecticide may be used. Similar considerations apply for liquid bait or foam bait and the concentration of IGR or larvicide or insecticide provided.

In a solid piece of bait, there may be at least about 0.01 mg to 0.1 mg of the IGR or larvicide or insecticide present for achieving the desired activity in a small pest animal. This would be enough to provide the equivalent of 0.5 mg to 10 mg per kilogram of weight for a 20 g mouse, for example, if the bait was to be wholly consumed by the mouse. In liquid or foam bait, a corresponding minimum concentration of IGR or larvicide or insecticide may be used, according to the expected foam exposure or liquid consumption of the pest.

For a medium-sized pest, such as a rat weighing up to 500 g, a quantity of about 0.1 mg to 1 mg of IGR (such as methoprene) or larvicide or insecticide may be suitable for absorption and distribution through the pest body. In an example of a liquid bait, a concentration of around 0.34 mg/liter might be used. For a 250 ml bottle, this would require only 0.085 mg of IGR such as methoprene per bottle. This is very small amount of active agent but can substantially mitigate the release of flies from a rodent carcass, for example.

In a solid piece of bait, there may be about 1 mg to 20 mg, or an integer value in that range, of the IGR (such as methoprene) or larvicide or insecticide. This amount may be suitable for a relatively large pest animal. This would be enough to provide the equivalent of 0.1 mg to 2 mg per kilogram of weight for a 10 kg raccoon, for example, if the bait was to be wholly consumed by the raccoon. In liquid or foam bait, a corresponding minimum concentration of IGR or larvicide or insecticide may be used, according to the expected foam exposure or liquid consumption of the pest.

Where the bait is a liquid or a foam containing the poison and the IGR and/or larvicide and/or insecticide, the IGR and/or larvicide and/or insecticide may be provided in a concentration of at least about 0.1 mg per liter. It will be appreciated that the amount of IGR/larvicide/insecticide used per unit volume of the liquid or foam may be determined based on the expected amount of bait to be ingested and the amount of IGR/larvicide/insecticide required to affect the target insect pest.

It is not envisaged that a particular maximum limit needs to be placed on the quantity or concentration of IGR and/or larvicide and/or insecticide included in the bait or composition. It will be appreciated there may be a limit in practical terms, such as the maximum amount used whilst keeping bait or composition palatable for a pest or accounting for the solubility of a compound, for example. In any case, the lowest amount which can achieve the desired effect is usually preferred for economic reasons. If a maximum limit was to be contemplated then an amount equivalent to 50 mg or 100 mg per kilogram of the pest may be appropriate.

The bait composition of the first aspect of the invention, or the bait of the second aspect of the invention, can be used for pest control of a first pest, such as one or more rodents, and also for the substantially concurrent or pre-emptive pest control of an insect pest, which would otherwise propagate via the carcass(es) of the rodent(s).

The bait composition of the first aspect of the invention, or the bait of the second aspect of the invention, can be provided in a suitable container. Examples of containers include a bottle, a tub, a packet, a box or a pest trap.

According to a third aspect of the invention, there is provided a method of pest control for an animal pest and for an insect pest which can propagate using a carcass or carcasses of the animal pest, comprising the step of: (a) deploying bait of the second aspect of the invention in the vicinity of a pest infestation site, for ingestion by the animal pest. The method may consist solely of this step, without the need for subsequent insect pest control.

The advantages are similar to those discussed for the first and second aspects of the invention. In particular, the invention enables pest control of two different pests in a single baiting step. That is, control of a primary target pest population and also of a second target pest population which normally proliferates after the death of some or all of the primary target pest population. This substantially mitigates the likelihood of needing further pest control for the secondary pest after baiting, for example to deal with a fly or other pest infestation after laying bait for rodents. That is to say, fewer flies and other invertebrate pests will result from dealing with the primary pest infestation, and there will be less consequential damage to human health and/or reputation of a person or business.

The method may include the step of: (b) monitoring or estimating the number of members of the insect pest present in the vicinity of the site, prior to or around the same time as performing step (a).

The method may include the step of: (c) between about three to ten days after step (a) has been performed, monitoring or estimating the number of members of the insect pest present in the vicinity of the site for assessing whether further pest control or bait testing (possibly targeted at just the insect pest) is required.

This allows the prevalence of insect pests before and after baiting to be monitored, in order to determine whether additional pest control measures for the insect pest are warranted.

This may also be useful to determine and/or optimize the amount of IGR, larvicide, insecticide and/or any other constituents of the bait for particular pest combinations. In that case, the method may be carried out multiple times using various amounts of active ingredients to ascertain the amounts required to substantially minimize insect propagation resulting from animal pest control. The method may in that case be considered to be a method of determining a bait composition for combined (or single step) pest control of an animal pest and a corresponding insect or invertebrate pest.

According to a fourth aspect of the invention, there is provided a non-toxic bait intended for ingestion by a pest, the bait comprising an insect growth regulator (IGR) for minimizing or controlling the propagation of insects such as flies from the pest's carcass when the pest is dead, and a carrier (e.g. liquid) or edible body comprising the IGR. In particular, there is provided a non-toxic bait as set out in claim 14. Optional features are set out in the dependent claims.

The term “non-toxic bait” is intended to mean a lure which is specifically intended to be eaten by a pest, and is used accordingly.

According to a fifth aspect of the invention, there is provided a method of pest control for a pest and for one or more insect species, such as one or more fly species, which can propagate using one or more carcasses of the pest. The method comprises the steps of: (a) providing non-toxic bait according to the fourth aspect in one or more locations which include a pest population, for ingestion by the pest; and after at least some of the non-toxic bait has been ingested by the pest, at least one of: (b) providing bait according to the second aspect, or other bait or poison which is toxic to the pest (such as aluminum phosphide tablets or cyanide tablets), in the one or more locations for ingestion by the pest to kill the pest; (c) using gas in the one or more locations to kill the pest; (d) providing one or more traps for the pest in the one or more locations, optionally with bait (such as the bait in (b)) or a lure disposed in the or each trap for attracting the pest; and (e) partially or wholly collapsing, or otherwise destroying, one or more burrows or nests for trapping and/or killing any pests therein.

The advantages of the non-toxic bait in the fourth and fifth aspects of the invention are similar to those of the first and second aspects of the invention. Any feature of those aspects may be included in the non-toxic bait, save for the pest poison.

In this case, there is no poison to gradually or immediately kill the animal pest that eats the bait. Instead, the operational principle is that the pest population eats the non-toxic bait and is ‘primed’ with IGR. This way, if the pest dies before an instance of pest control intended to kill the pest, then the residual IGR in its system should still substantially prevent adult insects or flies from hatching in or near the carcass.

In other words, whilst there is either a bifurcation of the IGR-doping and poisoning steps (if pest control kills the pest) or an absence of a poisoning step (e.g. if the pest dies of natural causes), the advantages of the invention are still realized.

Omitting a poison from the bait also mitigates unintentional poisoning of other non-target animals.

The non-toxic bait can be used for a first stage of pest control, to be followed in a second stage by e.g. use of toxic bait, such as bait according to the first or second aspects of the invention, or a conventional poison bait.

A suitable amount of time should be allowed between the stages to ensure that the non-toxic bait has been consumed by the pest to provide the IGR in its body. The timeframe may vary depending on when and to what extent the bait is ingested, but typically the timeframe will be on the order of days or weeks.

In other scenarios, the second stage of pest control may not use a bait but could involve deploying some sort of ‘area effect’ poison or toxin, or use of a trap or traps to capture the pest. For example, a gas which is poisonous to the pest may be used. A gas such as carbon dioxide may be used to effectively suffocate the pest, by displacing oxygen.

This can be useful in outdoor areas, such as a camp containing tents, shacks or other such temporary structures, rather than permanent buildings. Whilst such camps are usually set up with the intention of being temporary, they can sometimes remain occupied for months or even years, e.g. during disaster recovery efforts and re-building. Pests are inevitably attracted in the area, often due to comparatively poor sanitation and waste management, and pest infestations can become rife.

By deploying non-toxic bait around area of any such infestation, the pest population will eventually consume at least some of the bait. Pest control action can then be taken to kill the pests without subsequently having large numbers of insect pests (particularly flies) propagate from the carcasses as a result.

Preferably, where the pest population is distributed over a relatively large area (not just one residence), a gas is used to kill the pests. This is useful to kill pests in burrows with relative ease. For example, carbon dioxide is heavier than air and can quickly incapacitate and kill a pest such as a rodent.

A toxic or poisonous gas may be used. For example, cyanide gas or phosphine gas (e.g. generated from aluminum phosphide) may be used. However, any other suitable gas which kills the pest may be used.

The non-toxic bait may be provided as part of a kit with the toxic bait of the first/second aspect. The non-toxic bait may be provided in a separate container.

According to a further aspect of the invention, there is provided a bait composition for ingestion by a pest, the bait composition comprising a poison for killing the pest, and an insect growth regulator (IGR) for minimizing or controlling the propagation of an insect pest from the pest's carcass.

Any aspect of the invention may independently include any feature or features described with respect to any other aspect of the invention.

Further areas of applicability will become apparent from the description provided herein. The description and specific examples in this summary are intended for purposes of illustration only and are not intended to limit the scope of the present disclosure.

### DRAWINGS

For a better understanding of the present invention, and to show more clearly how it may be carried into effect, reference will now be made by way of example only to the accompanying drawings, in which:

FIG. 1 shows a flowchart of a process for carrying out pest control of a first pest via baiting and monitoring the effect of the pest control on numbers of a second pest; and

FIG. 2 shows a flowchart of a process for carrying out pest control of a first pest via a first non-toxic baiting step and a subsequent pest-killing or pest-capturing step.

### DETAILED DESCRIPTION

In some preferred embodiments, there is a bait composition for ingestion by a rodent, causing it to die, and which adversely affects the maturation of blow flies which consume the rodent's carcass. The bait composition is provided as part of solid piece of bait or pellet in some embodiments, or as part of a liquid or foam bait in other embodiments. The bait composition is distributed substantially evenly through the solid piece of bait or the liquid/foam bait. The bait or composition may be provided in a suitable sealable container or dispenser.

The bait includes a poison in the form of a rodenticide for killing a rodent. Any suitable rodenticide may be used, such as an anticoagulant or a compound for causing hypercalcemia in a rodent. The bait is therefore toxic to the rodent once ingested. If a naturally-occurring rodenticide is desired, then calcipherol and/or cholecalciferol may be included.

The bait also includes a feed-through insect growth regulator (IGR). Any suitable IGR which inhibits blow fly maturation from a larval stage may be included, such as methoprene, hydroprene or an azasteroid for example. If a naturally-occurring IGR is desired, then Egyptian essential oils or azadirachtin may be preferred. The IGR is selected to target and adversely affect metamorphosis of the insect pest at issue.

In some embodiments, the IGR may be supplemented by or replaced by a feed-through larvicide. Any suitable larvicide may be used, e.g. a DOPA decarboxylase inhibitor (such as methyl DOPA). The larvicide may include a pathogenic fungus for killing larvae in other embodiments.

In some embodiments, the IGR may be supplemented by or replaced by a feed-through insecticide. Any suitable insecticide may be used, e.g. permethrin.

The bait includes a pest attractant. For example, in the case of solid bait, the bait includes grain or another food sought after by rodents. In the case of liquid bait, the water in which the bait composition is dissolved or suspended may be sufficient as an attractant. If not, a sugar or sweetener may be added to both attract the rodent and potentially disguise any unusual flavor which may be caused by the bait composition.

In the solid bait, the IGR and/or the larvicide and/or insecticide are encapsulated in a biodegradable material to improve the longevity of the bait and/or minimize anti-feedant properties in some embodiments. This may not be appropriate in the liquid or foam bait, but the IGR and/or larvicide and/or insecticide can be provided in another protected form, such as by using suitable protecting groups which can be detached during digestion and uptake by the rodent. However, the IGR will in general be stable in the environment and during digestion, so a protected form may not be needed.

The amount of IGR and/or larvicide and/or insecticide is tailored to the expected weight of the pest, in this case a rodent. Typically, a rodent may weight anywhere between 10 g and 500 g, so for a solid piece of bait to be completely consumed by the rodent, a suitable amount of IGR in the bait may be between 0.01 mg to 1 mg, for example. It is preferred to select the amount of IGR such that the post-mortem amount of IGR in the rodent is around 1 ppm to 10 ppm. The equivalent dose in liquid or foam form will depend on the amount of liquid ingested by the rodent, but will typically be at least 0.1 mg per liter.

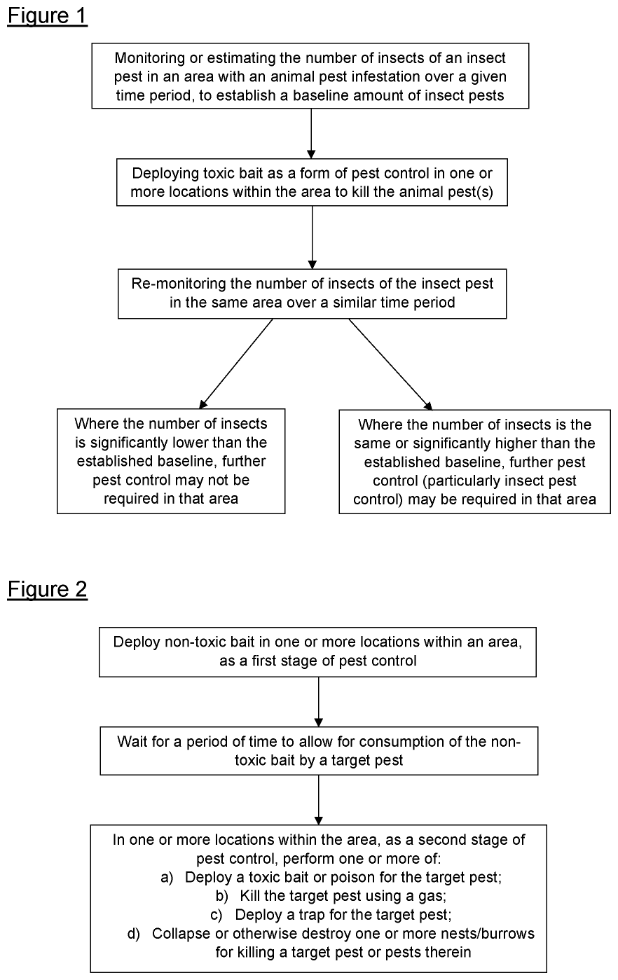

FIG. 1 sets out steps for an embodiment of a pest control method for two target pests—a primary pest, which is an animal pest such as a rodent (particularly a rat), and a secondary pest, which is an insect pest such as a blow fly. The death of the animal pest allows the insect pest to propagate or proliferate using the carcass of the primary pest. It should be noted that ascertaining the potential insect load for a particular carcass or infestation is not essential given that, once a mammal or bird dies, invertebrates will almost inevitably find the carcass, particularly in warm temperatures. However, it may be useful to determine the effectiveness of a particular bait, such as during bait testing.

In a first optional step, the number of pest insects for the secondary pest being targeted may be monitored or estimated shortly prior to bait deployment in the target area(s). This may in some embodiments instead be done immediately after bait deployment. The intention in either case is to perform the check before the bait has been deployed long enough to result in the death and start of decomposition of any of the target pests (the lag phase).

For example, this may involve monitoring the level of flies to establish an estimated baseline or background amount for the number of flies in the area(s) prior to dealing with a pest infestation. The approximate number of flies can be judged by using traps, typically involving chemical attractants (such as pheromones) or light tubes. The traps may incorporate either a funnel, one or more sticky boards or one or more zappers to allow for sampling and extrapolation. Other sorts of trap may be used for other insects.

In a second step, the bait containing the composition is used by deploying it in a target area or areas. In some embodiments, this includes leaving solid pieces of bait in a region or regions known or predicted to be frequented by the animal pest. In some embodiments, this includes leaving liquid bait (possibly in a container) in a similar region or regions, or applying a foam bait to surfaces the pest is expected to make contact with en route to and/or from its nest or territorial boundaries, for example.

Once the bait composition has been deployed in the target area(s), some time may pass before it is ingested by a member of the target pest animal group. However, assuming an infestation of at least one pest member, then the bait should eventually be ingested, through eating or drinking the bait or via grooming behavior. If the bait is not ingested (either at all or in a sufficient quantity) after a predetermined period of time, then it may need to be re-positioned or replaced entirely, or removed once no longer required to reduce the risk of non-target species being impacted. Liquid or foam bait may in any case need to be re-applied or re-deployed if it has evaporated, for example. Where possible, carcasses should be removed and disposed of accordingly.

Once ingested by the target pest, the poison is then absorbed, along with the IGR and/or larvicide and/or insecticide, via the pest's digestive system and spreads into its body tissues and organs. Some of each of the poison, the IGR, insecticide and/or the larvicide can also remain in its gastric tract and/or be excreted in the pest's feces, depending on the bait constituents and the particular pest animal involved.

The pest which ingested the bait will then die, typically a day or so after ingesting the bait. The pest can in some embodiments ingest the bait multiple times prior to death, if a slow-acting poison for that pest species is used.

Insects such as flies will then inevitably find the pest carcass and lay their eggs in it. The eggs hatch and the resulting larvae or maggots will consume the carcass, including the IGR, insecticide and/or larvicide. Where an IGR is present, this inhibits the development of the maggots into adult insects, causing them to perish in their pupae. Where a larvicide is present, some or all of the larvae die at the larval stage. Where an insecticide is present, some or all of the insects which visit or mature in the carcass should die from exposure to the insecticide.

In both cases, this limits the spread of insects from the carcass, which in turn limits the spread of the bait constituents to other non-target species, minimizing potential collateral environmental, health and hygiene issues, as well as potential reputational damage. Thus, the bait enables pest control of an existing pest (such as a rodent population), as well as enabling pre-emptive pest control of another pest (such as blow flies) which would potentially otherwise result from the first round of pest control.

In a third optional step, around 3 to 10 days after bait deployment, the number of insects for the insect pest can be checked again in the target area(s). Whilst this is optional, it allows pre- and post-treatment levels of insects to be monitored, in order to make a determination about whether additional pest control measures for the insect pest are needed.

If insect levels are zero or substantially lower (to a statistically significant extent) than the baseline recorded previously, this can indicate that the pest control (or testing) has been at least partly successful and no further measures may be required for the time being, or that the bait has a suitable composition for the particular animal and insect pest combination being monitored. Further monitoring may be prudent to determine whether the presence of any remaining insects can be attributed to a residual pest infestation in the same area or another pest infestation in a different area nearby.

Note that where the initial and subsequent monitoring involves outdoor monitoring of insects, it will be appreciated that some consideration must be given to weather conditions and the time of day monitoring is carried out. The parameters of each monitoring period and the equipment and techniques used should ideally be the same.

If insect levels are substantially higher (to a statistically significant extent) than the baseline recorded previously, or if insect levels are substantially the same and non-zero, this may imply that pest control has been ineffective, or that there is another infestation nearby. This may then lead to a decision to undertake further pest control (or testing), either in the same area(s) or another area or areas. The further pest control may be targeted for the insect pest if there is suitable evidence that the animal pest infestation has been dealt with, for example by monitoring bait consumption or disturbance.

Note that the time delay required for a second round of monitoring will depend on the pest species under consideration, and the efficacy of the bait used, and so the second round of insect pest monitoring can be brought forward or delayed accordingly.

It will be appreciated that other embodiments are envisaged in which the first target pest is a pest other than a rodent, and the poison identity and quantity are selected accordingly. The bait may be a generic bait for combination treatment of multiple pests in a particular region. In that case, the combination treatment may contain multiple IGRs to suit two or more respective target insect species.

It will also be appreciated that other embodiments are envisaged in which the second target pest is a pest other than a blow fly, and the IGR and/or larvicide and/or insecticide identity and quantity are selected accordingly.

In further embodiments of the invention, there is a non-toxic bait for ingestion by a rodent. The non-toxic bait has an edible body, such as a bait block, which contains a suitable amount of IGR. In other embodiments, the non-toxic bait may have a carrier liquid or carrier foam which contains the IGR.

The non-toxic bait includes similar features to those discussed for the above embodiments. Any of the features in preceding embodiments may be included in isolation from other features or in combination with other features, with the exception that the non-toxic bait does not itself contain a poison for killing the pest.

FIG. 2 sets out steps for an embodiment of a pest control method for two target pests—a primary pest, which is an animal pest such as a rodent (particularly a rat), and a secondary pest, which is an insect pest such as a fly. As discussed with respect to FIG. 1, the death of the animal pest allows the insect pest to propagate or proliferate using the carcass of the primary pest.

The purpose of the non-toxic bait in the method is to introduce IGR into an animal pest population, such as a rodent or rat population, where the pest population is distributed across an area as opposed to localized in a particular residence.

The first stage of the method involves deploying or providing the non-toxic bait in one or more locations in or near the vicinity in which the pest population is located (or believed to be located). That is, stage one is a ‘priming’ phase where IGR is doped into the pest population.

Once the non-toxic bait has been laid out, it is necessary to wait for a period of time until the bait has been at least partly consumed by the target animal pest. This may be checked by assessing bite marks on the bait or using camera traps for example. In some cases, it may be possible to use bait stations to limit bait access by various non-target pests, but the scale of the pest infestation may mean that this is not practical.

The period of time involved may be anything from a couple of days to a few weeks, for example. If it is known or anticipated that there is a relatively large pest population, multiple rounds of non-toxic bait may be needed to maximize the likelihood that most or all of the pest population has ingested the bait.

The next stage of the method is the ‘extermination’ phase. One means of doing this is to provide or deploy conventional poison bait or toxic bait as described in earlier embodiments. This kills the pests and their carcasses are decomposed in the same manner as previously described. Multiple rounds of toxic baiting may be required.

Another option is to actively apply or direct gas into and/or around the locations where the pest population is known or believed to be. This may involve temporarily evacuating the local human population, if the gas is toxic to humans. It is particularly useful where the pests live or hide in inaccessible locations such as burrows. Multiple rounds of gassing may be required. Carbon dioxide gas may be used, or any suitable gas which achieves the aim of killing the pests. This can include a cyanide gas or phosphine gas, e.g. evolved when a pest bites into a corresponding tablet, for example.

Another option is to deploy one or more traps to capture the pests. Toxic bait may be provided in the traps to kill the pests.

Another option is to partially or wholly collapse the burrow(s) where the pest may be located. The burrow entrance may be collapsed to trap the pest inside, optionally after delivering bait and/or gas into the burrow(s). Further collapse or destruction may be appropriate depending on where the nest or burrow is. This traps the pest inside so that it perishes, or may kill the pest instantly depending on the extent of burrow/nest collapse.

A combination of two or more (or all) of these approaches may be used to maximize the extent to which a pest population is reduced or completely eliminated. For example, the traps may be used to tackle residual pest population that is not killed via gassing.

It will be appreciated that the non-toxic bait version of the invention can therefore be used to achieve a similar result to the toxic bait version, despite the separation of the poisoning/killing/death from the introduction of IGR into the pest.

It will be appreciated that equivalent larvicide-based and/or insecticide-based versions of the non-toxic bait may be provided and used in a corresponding manner.

The embodiments described above are provided by way of example only, and various changes and modifications will be apparent to persons skilled in the art without departing from the scope of the present invention as defined by the appended claims.

What is claimed is:

1. A bait composition for ingestion by a pest, the bait composition comprising:
a poison for killing the pest to produce a pest carcass; and
an insect growth regulator (IGR) which targets fly larvae at the third or fourth instar for minimizing or controlling the propagation of flies from the pest carcass, the IGR being suitable for uptake into body tissues and organs of the pest following ingestion of the bait composition by the pest during use, and the IGR being provided in an amount which after ingestion provides an IGR concentration which is at least 1 ppm in the body tissues and organs of the pest carcass but which is insufficient to kill fly larvae prior to pupation, in use allowing fly larvae to decompose the pest carcass but thereafter substantially preventing flies from hatching from fly pupae developed from larvae in the pest carcass.

2. The bait composition of claim 1, wherein the pest is a rodent, and the poison includes a rodenticide.

3. The bait composition of claim 1, wherein the insect growth regulator includes one or more compounds independently selected from the following group: methoprene, hydroprene, diflubenzuron, tebufenozide, RH 5849, pyriproxyfen, chromafenozide, methoxyfenozide, diofenolan, fufenozide, and lufenuron.

4. The bait composition of claim 1, wherein the insect growth regulator includes one or more components independently selected from the following group: Egyptian essential oil(s), celery, fenugreek, mustard, rosemary, anise, lettuce, and chamomile, or an extract thereof.

5. The bait composition of claim 1, wherein the insect growth regulator includes one of more of: azadirachtin, neem extract, and Persian lilac or chinaberry tree extract.

6. The bait composition of claim 1, wherein the insect growth regulator includes an azasteroid for inhibiting one or more of sterol uptake, transport and use in a fly larva.

7. The bait composition of claim 1, wherein the insect growth regulator is encapsulated in a biodegradable material.

8. The bait composition of claim 1, wherein the insect growth regulator includes one or more of: Egyptian essential oil(s), celery, fenugreek, mustard, rosemary, anise, lettuce, chamomile, azadirachtin, neem extract, and Persian lilac or chinaberry tree extract; and in which the poison includes one or both of ergocalciferol and cholecalciferol for providing a natural bait composition.

9. Bait for ingestion by a pest, comprising the bait composition of claim 1.

10. The bait of claim 9, wherein the insect growth regulator is selected and the amount thereof is tailored according to one or more of: the pest the bait is intended for, the fly or flies expected to use the pest carcass for their larvae, and the mass of the bait.

11. The bait of claim 9, wherein the bait is in the form of a substantially solid body such as pellet, a grain, or a block, and the poison and the insect growth regulator are distributed substantially homogenously throughout the solid body.

12. The bait of claim 11, wherein the insect growth regulator is provided in the solid body in an amount of about 0.01 mg to about 50 mg, or about 0.01 mg to about 20 mg.

13. The bait of claim 9, wherein the bait is a liquid or a foam containing the poison and the insect growth regulator, and the insect growth regulator is provided in a concentration of at least about 0.1 mg per liter.

14. Non-toxic bait intended for ingestion by a pest, the bait comprising:
a carrier or edible body; and
an insect growth regulator (IGR) which targets fly larvae at the third or fourth instar for minimizing or controlling the propagation of flies from a pest carcass when the pest is dead, the IGR being suitable for uptake into body tissues and organs of the pest following ingestion of the bait by the pest during use, and the IGR being provided in an amount which after ingestion provides an IGR concentration which is at least 1 ppm in the body tissues and organs of the pest but which is insufficient to kill fly larvae prior to pupation, thereby when the pest is dead allowing fly larvae to decompose the pest carcass but thereafter substantially preventing flies from hatching from fly pupae developed from larvae in the pest carcass.

15. The bait of claim 14, wherein the insect growth regulator includes one or more of the following: methoprene, hydroprene, diflubenzuron, tebufenozide, RH 5849, pyriproxyfen, chromafenozide, methoxyfenozide, diofenolan, fufenozide, lufenuron, Egyptian essential oil(s), celery, fenugreek, mustard, rosemary, anise, lettuce, chamomile, azadirachtin, neem extract, Persian lilac or chinaberry tree extract, or an azasteroid for inhibiting sterol uptake, transport and/or use in a fly larva.

16. The bait of claim 14, wherein the bait is in the form of a substantially solid body such as pellet, a grain, or a block, and the insect growth regulator is distributed substantially homogenously throughout the solid body, in which the insect growth regulator is provided in the solid body in an amount of about 0.01 mg to about 50 mg, or about 0.01 mg to about 20 mg.

17. The bait of claim 14, wherein the bait is a liquid or a foam containing the insect growth regulator, and the insect growth regulator is provided in a concentration of at least about 0.1 mg per liter.

18. The bait of claim 14, wherein the bait is used for pest control of a pest and also substantially concurrent or pre-emptive pest control of flies.

19. The bait of claim 9, provided in a container.

20. A method of pest control for a pest and for one or more fly species which can propagate using one or more carcasses of the pest, the method comprising the steps of:
a) providing non-toxic bait according to claim 14 in one or more locations which include a pest population, for ingestion by the pest; and
after at least some of the non-toxic bait has been ingested by the pest, one or more of:
b) providing toxic bait or poison in the one or more locations for ingestion by the pest to kill the pest;
c) using gas in the one or more locations to kill the pest;
d) providing one or more traps for the pest in the one or more locations, optionally with bait or a lure disposed in the or each trap for attracting the pest; and
e) partially or wholly collapsing, or otherwise destroying, one or more burrows or nests for killing any pest therein.
